(19) United States
(12) Patent Application Publication
(10) Pub. No.: US 2005/0192481 A1
(43) Pub. Date: Sep. 1, 2005

(54) Laryngoscope and camera coupling

(76) Inventors: George Berci, Los Angeles, CA (US); Marshal B. Kaplan, Beverly Hills, CA (US); James P. Barry, Charlton, MA (US); David Chatenever, Santa Barbara, CA (US); Klaus M. Irion, Liptingen (DE); Andre Ehrhardt, Tuttlingen (DE); Jurgen Rudischhauser, Tuttlingen (DE); Daniel Mattsson-Boze, Sacramento, CA (US)

Correspondence Address: ST. ONGE STEWARD JOHNSTON & REENS, LLC, 986 BEDFORD STREET, STAMFORD, CT, 06905-5619, US

(21) Appl. No.: 11/062,131

(22) Filed: Feb. 18, 2005

Related U.S. Application Data
(63) Continuation-in-part of application No. 10/739,670, filed on Dec. 18, 2003, now Pat. No. 6,875,169.
Application No. 10/739,670 is a continuation-in-part of application No. 10/285,190, filed on Oct. 31, 2002, now Pat. No. 6,890,298.
Application No. 10/285,190 is a continuation-in-part of application No. 09/418,542, filed on Oct. 14, 1999, now Pat. No. 6,494,826.

(51) Int. Cl. A61B 1/267

(52) U.S. Cl. 600/188; 600/197

(57) ABSTRACT

A laryngoscope with a detachable camera, the system providing illuminating light to and images of the area ahead of the laryngoscope to facilitate insertion of the laryngoscope without damage to the surrounding tissue, the system including a keyed attaching device for facilitating attachment of camera and laryngoscope in a correct orientation.

### Priority Document

This is a continuation-in-part of U.S. patent application Ser. No. 10/739,670 filed Dec. 18, 2003, which is a continuation-in-part of U.S. patent application Ser. No. 10/285,190 filed Oct. 31, 2002, which is a continuation-in-part of U.S. Pat. No. 6,494,826 filed Oct. 14, 1999.

### FIELD OF THE INVENTION

The invention relates to a video system and more specifically to a video laryngoscope with a light and image guiding system positioned within a blade of the laryngoscope and a camera unit that is detachably connectable the laryngoscope.

### BACKGROUND OF THE INVENTION

In the United States, approximately 10 million patients are anesthetized and intubated each year. While anesthetized, the patient's breathing functions are temporarily disabled. Ventilation is therefore supplied to the patient by the anesthesiologist during the procedure.

Ventilation is provided through an endotracheal tube. This tube is inserted into the trachea, and it is closed against the wall of the trachea by an inflatable cuff. The insertion of this tube involves risks that the anesthesiologist seeks to avoid or at least minimize. It is estimated that between one in 6,000 to one in 8,000 general anesthesia procedures result in death. There are of course many causes but of these it is estimated that about one third of them are caused by the intubation procedure.

One of the foremost obstacles encountered by the anesthesiologist in the intubation process include: the remoteness of the location where the tube is to be positioned, the consequent restriction of view as the tube is inserted, variations and anomalies in the anatomy of the patients, an uncomfortable and unnatural position for the anesthesiologist while holding the instrument, the potential need to change blades during the procedure, and the necessity for rapid intubation.

During the intubation process, it should be noted that when the tube is inserted, the patient is asleep, hyperoxygenated and then paralyzed for the procedure, and therefore not breathing. In addition, the ventilator is not yet in operation. This gives the anesthesiologist only about two minutes in which to intubate the patient, inflate the cuff, and start ventilation. If he is delayed because of unsuccessful attempts, he must stop, apply a ventilation mask to the patient, supply oxygen for a time through the mask, remove the mask, adjust medication if necessary, and then start over again. This delays the operation and extends the patient's time under anesthesia. This extension of time while under anesthesia and repeated attempts may have very serious consequences, especially for elderly patients.

With the advent of endoscopic equipment and small cameras, instrumentation has been improved to the extent that it can enable viewing of the cords and larynx on a video screen thereby facilitating the intubation of the patient in a relatively quick and safe manner. However, laryngoscopes and endoscopes may be further improved such that the laryngoscope is easier to use, thereby reducing the time involved in, for instance, changing a camera or light and image attachment.

Video laryngoscopes and endoscopes typically contain a removable light guiding system, usually in the form of fiber optic cables, in order to bring light to the surgical area. The light guiding system typically extends through the handle of the laryngoscope and through a guide tube located in the blade so as to position the light guiding system to illuminate the area ahead of the blade. Video laryngoscopes and endoscopes also typically contain an image guiding system, for example in the form of a rigid rod lens or flexible image transmitting system. The image guiding system can also be configured as an ordered, flexible fiber optic bundle. With a laryngoscope, the image guiding system is utilized to transmit reflected light and image from the area ahead of the blade to a camera. The camera, attached at the proximal end of the endoscope, usually contains a CCD (charge coupled device) sensor, in the form of a light-sensitive chip that converts the optical signals into electrical signals that are conveyed from the image-sensing camera module to a remotely located image processing system. The image guide typically extends from the distal end of the blade through the guide tube and then through the handle of the laryngoscope.

Typically, the combination light and image guiding system are permanently attached to the handle and are continuous, extending from the distal end of the blade, through the handle of the laryngoscope and to the camera for the image guiding system, and to the light source for the light guiding system. Typically the blade is removable from the handle, and therefore the light guiding system and image guiding system extending from the handle of the laryngoscope for insertion into the guide tube of the blade typically comprise flexible coherent fiber optic bundles. When changing blades, the bundle must be carefully inserted or withdrawn from the opening of the guide tube at the proximal end of the blade. This may take an unacceptable amount time for the physician to thread the bundle into the tube if the blade must be changed in the middle of the intubation process.

Another problem with known systems is that, the flexible bundles may easily be damaged and will wear over time, degrading or rendering the system inoperable. As a visual inspection of the device often will not indicate whether the bundles are damaged, it is conceivable that a physician may obtain a damaged or malfunctioning laryngoscope not realizing that it is damaged. The time involved with determining that the instrument is malfunctioning, withdrawing it, finding another laryngoscope, and then intubating the patient may have severe adverse effects upon the patient under anesthesia.

It is therefore desired to provide an improved video laryngoscope system with a highly durable illuminating light and image guiding system in which the fiber optic bundles need not be removed from the laryngoscope.

It is further desired to provide a simplified video laryngoscope that is easy to use and has fewer removable components, thus requiring a minimal amount of assembly and disassembly.

It is further desired to provide an improved video laryngoscope that will reduce the costs associated with the manufacture.

It is also desired to provide an improved video laryngoscope having a coupling mechanism that will reliably connect the laryngoscope the light and image guides to a camera unit while requiring a minimal amount of attention from the user to attach or detach.

### SUMMARY OF THE INVENTION

These and other objectives are achieved by providing a video laryngoscope having a handle permanently fixed to a blade, illuminating light and image guides or bundle fixed within or to the blade, and a coupling mechanism for rigidly connecting the laryngoscope handle to a camera unit.

The laryngoscope handle is provided with a cavity for receiving the camera unit therein. The cavity is provided with a coupling mechanism or coupling element to engage with a corresponding coupling mechanism or coupling element located on the camera unit.

The fixed blade, which is attached at one end to the laryngoscope handle, includes the light and image guides for transmitting illuminating light to and transmitting reflected light from the area ahead of a distal end of the blade. The bundle is fixed within the blade and extends approximately from the distal end of the blade to the coupling element within the cavity of the handle. The illuminating light and image guides located in the laryngoscope are in communication with corresponding illuminating light and image guides provided in the camera unit via the coupling element.

Accordingly, the coupling element is provided with a first stem having a diameter and length, within which a proximal end segment of the illuminating light guiding system is positioned, and which projects from one coupling end of the light and image guiding attachment in the coupling direction. Further, a second stem is provided whose length and diameter are greater than the length and diameter of the first stem, having a proximal end segment of the image guiding system being received in the interior of the second stem, and which projects from one coupling end of the light and image guiding attachment in the coupling direction. The second stem coacts with an interlock system arranged in the camera unit forming a rigid mechanical coupling, the first and second stems extending at a distance next to one another. Complementary receptacles corresponding to the two stems, into which the stems penetrate, are provided in the camera unit.

The mechanical, light-guiding, and image-guiding coupling is accomplished by way of a single simple linear displacement operation, in which specifically the two stems are pushed into the corresponding receptacles within the laryngoscope handle. Because one of the two stems is thicker and longer than the other, incorrect (i.e. reversed) insertion is not possible. Because the thicker stem is also simultaneously the longer one, it is possible, without undue attention, to feel for the correspondingly larger receptacle in the camera unit with this thicker and longer stem, and then to close the coupling with an insertion movement. Incorrect attachment is thus no longer possible, since the thicker and longer stem cannot be attached to the smaller-diameter receptacle for the smaller and shorter stem.

The mechanical interlock or coupling is affected simultaneously with this insertion. Because the larger stem is also the longer stem, and it carries the image guiding system, the image-guiding connection occurs at an axial spacing from the light-guiding connection. This feature has the advantage that any stray light that might emerge from the light connection cannot directly come into contact with the image-guiding connecting point located at an axial distance therefrom. The disadvantages of connecting image and light at the same level, or those, for example, of a coaxial arrangement, are thus eliminated.

Because the coupling mechanism is keyed, the physician can therefore, for example, sense the coupling element and its precise grasped position in the coupling region with one hand, and with the other hand can easily sense the camera unit and its grasped position as well, so that the two elements to be coupled can then be inserted into one another without visual contact. This greatly facilitates handling, especially when, during a procedure such as an intubation, one blade must be quickly exchanged for another thereby requiring that the light and image guiding attachment be removed and re-attached along with the new blade.

An interlock system is displaceable transversely to the coupling direction that can be engaged into a recess on the second stem. This feature has the advantage that in order to close and/or release the coupling, the locking element is displaced transversely to the coupling direction and is engaged into or disengaged from the recess on the second stem. These are all procedures that can be controlled, without visual contact, with the fingers of one hand; the snapping of the locking element into and out of the recess on the stem indicates to the operator whether the coupling is closed or open. If the locking element needs to be pushed into the recess, for example to close the coupling, this can be done by simply inserting the stems into their corresponding receptacles; precise locking can be ascertained by an audible sound that the locking element has been engaged. The locking element may comprise for instance, ball catches, hooks, snap lugs, or the like.

The locking element is acted upon by the force of a spring, and radially projects into the receptacle for the second stem. This is advantageous because, the force of the spring presses the locking element into a defined position, and the coupling may be disengaged by the application of a force opposite the coupling direction, namely withdrawing the stems from their respective receptacles. These are all procedures that can be sensed and controlled with the hand's sense of touch, so that no visual attention or observation is necessary when closing and opening the coupling.

The second stem may have a conical segment at the end that is followed by an undercut. The conical segment constitutes an insertion aid upon insertion of the stem into the receptacle, so that exact insertion is guaranteed with even approximate placement. At the same time, the conical surface can be utilized to displace the locking element radially upon insertion.

In addition, the undercut in the second stem may be configured as an annular groove. This forms a relatively large engagement surface with the locking element, so that the mechanical forces acting on the coupling will be dispersed over the entire area, which contributes to mechanical stability and less wear through use.

In addition, the first and second stems along with the receptacles receiving them each have a window. The windows thereby provide a sealed closure for the light and image guiding systems.

The camera unit is detachably connectable within the laryngoscope handle such that it may be detached when, for instance, the video laryngoscope is to be sterilized. The camera unit has, at one end, the coupling receptacles for the light and image guiding attachment, and at the other end an illumination cable connected to an illumination source and an image cable extending to a display screen. The illumination cable and the image cable may be either permanently attached to the camera unit or may be detachably connected.

While the camera unit has been described having receptacles located therein and the laryngoscope has been described provided with the stems, it is contemplated that these are interchangeable, namely the stems could easily be provide on the camera unit with the receptacles provided on the laryngoscope.

In one advantageous embodiment a laryngoscope system is provided comprising, a handle having a cavity located therein and a first light and image guiding attachment, and a blade connected to the handle. The system further comprises a camera detachably connectable to the handle and having a second light and image guiding attachment complementary to and engagable with the first light and image guiding attachment along a direction of movement. The system is provided such that the first light and image guiding attachment and the second light and image guiding attachment collectively include, an image guiding stem, engagable along the direction of movement with an image guiding receptacle, and a light guiding stem, engagable along the direction of movement with a light guiding receptacle.

In another advantageous embodiment a method for attaching a camera to a handle of a laryngoscope is provided comprising the steps of, providing a cavity within the handle, and providing a light guiding stem and an image guiding stem on the handle. The method further comprises the steps of positioning a camera within the cavity, the camera having a light guiding receptacle and an image guiding receptacle complementary to and engagable with the light guiding stem and the image guiding stem, and engaging the light guiding stem and the image guiding stem with the light guiding receptacle and the image guiding receptacle respectively along a direction of movement.

In still another advantageous embodiment a laryngoscope system is provided comprising, a handle having a first light guide and first image guide located therein, and a first light and image guiding connector associated with the handle. The system further comprises a blade connected to the handle and a second light guide and a second image guide located in the blade. The system still further comprises a second light and image guiding connector associated with the blade, the second light and image guiding connector being complementary to and engagable with said first light and image guiding connector along a direction of movement.

The laryngoscope system may in another embodiment also comprise a camera located in the handle that generates an image signal for display on a video screen which may be located in the vicinity of, remotely to or be attached directly onto the laryngoscope. It is further contemplated that the laryngoscope blade may comprise a permanently mounted blade to the handle, may be removable, and/or may be disposable. In any event, the light and image guides are located in the blade and are coupled to light and image guides located within the handle and may further be removably or non-removably positioned within the blade. In the event the blade is removable, the respective light and image guides may be coupled to each other via a connector having complementary parts located in the handle and the blade. The blade and handle may then be provided with a locking device for rigidly securing the blade to the handle.

It is still further contemplated that the laryngoscope system may be provided as a portable system having a portable power and illuminating light source located in, for instance, the handle.

It is understood that the features mentioned above and those yet to be explained below can be used not only in the respective combinations indicted, but also in other combinations or by themselves, without leaving the context of the present invention.

### BRIEF DESCRIPTION OF THE DRAWINGS

FIG. 1 is an illustration of a video laryngoscope system with a curved blade and fixed handle, and a light and image system engaged in the handle.

FIG. 2 is an illustration of light and image system having light and image guiding receptacles at one end and light and image cables connected to the other end, and a coupling element for attachment with the receptacles.

FIG. 3A is a perspective view in the cavity of the handle containing a coupling element for the light and image system attachment.

FIG. 3B is a cross-sectional view of the video laryngoscope containing a coupling element for the light and image system attachment.

### DETAILED DESCRIPTION OF THE DRAWINGS

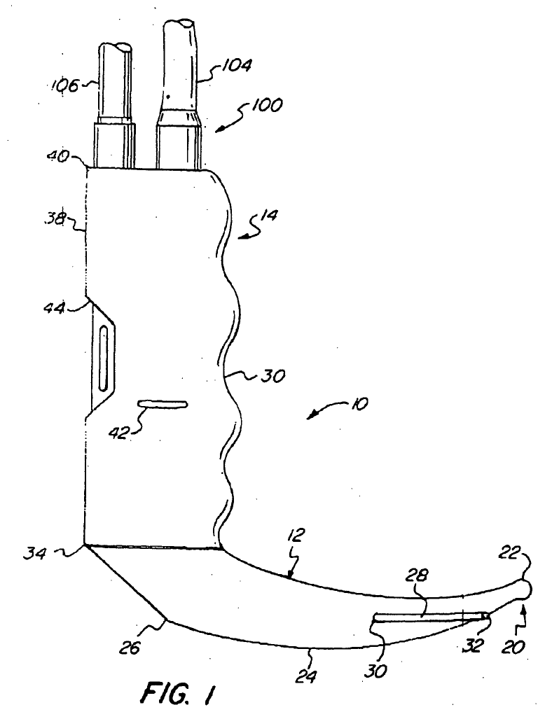

FIG. 1 shows an exemplary embodiment of a video laryngoscope 10 according to the present invention. The video laryngoscope 10 includes a blade 12 and a handle 14. The blade 12 may be of any size or type known to those of ordinary skill in the art. For example, the blade 12 may be a size 3 or 4 Mcintosh blade. The blade 12 has a distal end 20 which may be smoothed by a bulb-like edge 22. The blade 12 has a curved top surface 24 extending from the distal end 20 toward a proximal end 26. This top surface 24 may be used to elevate the tongue and permit visualization of the vocal cords beneath.

The blade 12 is typically fixed to the handle 14. In the exemplary embodiment, the proximal end 26 of the blade 12 is fixed to the handle at a joinder 34. The handle 14 typically includes a lower gripping surface 36 and an upper surface 38. The lower gripping surface 36 may include finger grips or contours for ease of handling. The handle 14 includes a cavity 40 for receiving a light and image system. For example, the cavity may receive a detachable camera unit 100. The upper surface 38 may include an opening 44 for access to a portion of the detachable camera unit 100.

The handle 14 may include one or more locking mechanisms 42 for securing the camera unit 100 in the handle 14. The locking mechanism 42 can comprise any appropriate mechanical interlocking system that may be engaged by insertion of the camera unit 100 into the handle 14, such as for instance, a ball detent or equivalent engagement means.

As shown in FIG. 1, the blade 12 further includes a bundle 28. The bundle 28 provides for the transmission of illuminating light and for the transmission of reflected light. The bundle 28 may include for instance, an image guide (not shown) and a light guide (not shown). The bundle 28 may optionally include a rigid outer shaft comprised of stainless steel or other non-corroding material. The bundle may further include a window 32, located at the distal tip of the bundle 28, to provide a seal for the image and light guides.

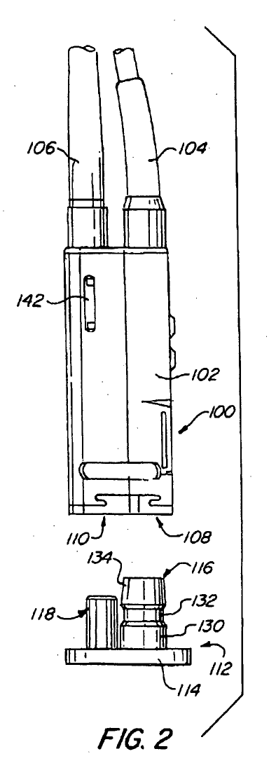

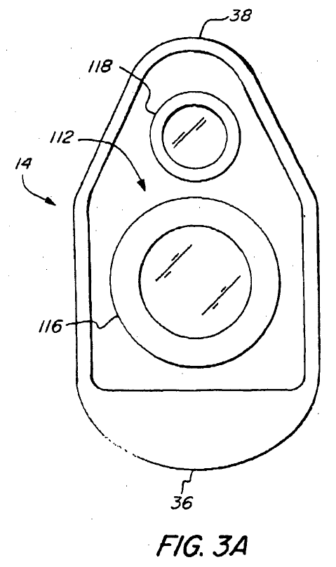

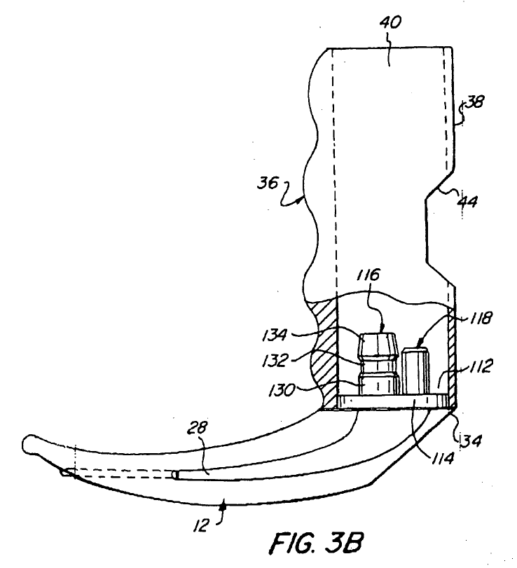

Shown also in FIG. 3B, the bundle 28 is fixed within the blade 12 of the video laryngoscope 10. The bundle 28 typically extends from a coupling element 112 (shown in FIG. 2) in the handle 14 and along the interior of the blade 12. In the exemplary embodiment, the bundle 28 extends within the blade 12 from the proximal end 26 and in an approximate direction towards the distal end 20. The bundle 28 protrudes via a hole 30 in the blade 12 (e.g., on the top surface 24).

FIG. 2 shows the camera unit 100 (i.e., light and image system) and the coupling element 112. The camera unit 100 customarily comprises a CCD chip and has a housing 102 that is generally rectangular but may be any shape. Furthermore, the shape of the housing 102 may be keyed to a cavity 40 of a video laryngoscope 10 as to only allow insertion of the camera unit 100 in a particular orientation. At one end, an image guiding cable 104 and a light guiding cable 106 are connected to the camera unit 100.

Image guiding cable 104 may comprise an electrical cable, which extends from the camera output (not shown), to a video display (not shown). Image guiding cable 104 may utilize any suitable format and protocol for the transmission of video images. Image guiding cable 104 may be permanently attached to the camera unit 100 as illustrated in FIG. 2, or may be detachably connectable to camera unit 100.

Also shown attached to the camera unit 100 is light guiding cable 106. Light guiding cable 106 may comprise any suitable cable (typically fiber optic) for the transmission of illuminating light from an illumination source (not shown) to a location to be illuminated. Light guiding cable 106 may comprise coherent or non-coherent fiber optic cables and be permanently attached to the camera unit 100 as shown in FIG. 2, or may be detachably connectable to the camera unit 100. While image guiding cable 104 and light guiding cable 106 are shown separate from each other, it is contemplated that both may be enclosed in a protective jacket as a single cable.

In an exemplary embodiment of the present invention, the image guiding cable 104 and the light guiding cable 106 may connect to a compact portable unit. The compact portable unit may comprise for instance, a camera control unit, a light source and a video display screen positioned convenient to the physician.

At the end opposite to where image guiding cable 104 and light guiding cable 106 attach to camera unit 100, image guiding receptacle 108 and light guiding receptacle 110 are provided. Also provided on camera unit 100 is a recess 142, which engages the locking mechanism 42, provided in the handle 14.

As shown in FIG. 2, housing 102 of camera unit 100 is contoured, which allows easy gripping and manipulation of camera unit 100 by a physician even with having to look at the device. Rather, based on the contour of the housing 102, a physician will be able to determine by feel, how to position camera unit 100 to connect it with various pieces of equipment. For example, the cavity 40 in the handle 14 is similarly contoured to accept the camera unit 100 in a single orientation only. Therefore, the camera unit 100 may be easily inserted and removed from the handle 14 with having to look at the device. Housing 102 may comprise any rigid material, such as for instance, stainless steel, that will protect the camera unit in a cost effective and durable manner.

Also illustrated in FIG. 2 is coupling element 112. Coupling element 112 comprises coupling element housing 114, along with image guiding stem 116 and light guiding stem 118. The light guiding system extending through light guiding stem 118 comprises a bundle of coherent fiber optic cables for transmission of reflected light. Coupling element housing 114 may be any shape or size to suit the particular application and may comprise any suitable rigid material, such as for instance, stainless steel or rigid plastic. Coupling element 112 is designed to detachably engage with image guiding receptacle 108 and light guiding receptacle 110.

While coupling element 112 is illustrated in FIG. 2 is not specifically shown connected to a particular instrument, it may be used in connection with practically any medical instrument using an illuminating system and an image guiding system. The coupling mechanism is universal and may be easily adapted depending on the quality and resolution of image desired. For example, coupling element 112 may be used in connection with the video laryngoscope 10 according to the present invention.

FIGS. 3A and 3B show coupling element 112 utilized in the video laryngoscope 10 according to one advantageous embodiment of the present invention. FIG. 3A shows a perspective view in cavity 40 of handle 14, and FIG. 3B shows a cross-sectional view of an exemplary embodiment of video laryngoscope 10. As shown, the handle 14 typically includes a coupling element 112 attached within cavity 40. The camera unit 100 may be inserted in the cavity 40 (as shown in FIG. 1) to detachably connect with the coupling element 112. Therefore, both image guiding stem 116 and light guiding stem 118 extend in a coupling direction to mate with image guiding receptacle 108 and light guiding receptacle 110 respectively. As shown in FIG. 3B, image guiding stem 116 comprises a cylindrical segment 130, and annular groove 132, and a terminal conical segment 134.

Referring back to FIG. 2, a locking mechanism (not shown) may be located in the housing 102, displaceable radially with respect to the coupling direction. In particular, the locking mechanism may be located in the imaging guiding receptacle 108 of the housing 102. Conical segment 134 of image guiding stem 116 thereby encounters the locking element projecting into image guiding receptacle 108 and displaces them radially outward. When image guiding stem 116 has been pushed into image guiding receptacle 108 to the point that the ends of the locking element come to rest at the level of annular groove 132, they snap into annular groove 132. Thus, the camera unit 100 becomes interlocked to the coupling element 112. All that is necessary to release the coupling is withdraw the coupling element 112 outward with enough force to overcome the locking element as engaged in the annular groove.

Shown in FIG. 3B, bundle 28 is attached to the coupling element 112 in proximity to the joinder 34. The bundle 28 typically contains a light and image guide allowing for the transmission of illuminating light ahead of the blade 12 and for receiving reflected light (e.g., images) and transmitting it back to the camera unit 100.

As discussed above, the bundle is typically fixed within the blade 12. Advantages of this design include the lack of required assembly and disassembly for use. A physician must only insert the camera unit 100 to ready the video laryngoscope 10 for use. If a different blade is desired, a physician must only quickly remove the blade 10 and handle 14 assembly and reattach a alternate blade 10 and handle 14 assembly.

An additional advantage of the present invention is that disassembly of the video laryngoscope is not required for sterilization. The blade 12 and handle 14 assembly, including the bundle 28, may be sterilized in any immersion fluid (e.g., any FDA approved immersion fluid).

Although the invention has been described with reference to a particular arrangement of parts, features and the like, these are not intended to exhaust all possible arrangements or features, and indeed many modifications and variations will be ascertainable to those of skill in the art.

1. A laryngoscope system comprising:
a handle having a cavity located therein and a first light and image guiding attachment;
a blade connected to said handle;
a camera detachably connectable to said handle and having a second light and image guiding attachment complementary to and engagable with the first light and image guiding attachment along a direction of movement;
said first light and image guiding attachment and said second light and image guiding attachment collectively including:
an image guiding stem, engagable along the direction of movement with an image guiding receptacle; and
a light guiding stem, engagable along the direction of movement with a light guiding receptacle.

2. The system according to claim 1 further comprising a light guide and an image guide located within said blade.

3. The system according to claim 2 wherein said light guide and said image guide are in communication with corresponding light and image guides located in said camera via the light and image guiding attachments.

4. The system according to claim 1 wherein said handle includes a camera locking mechanism for securing said camera in said handle.

5. The system according to claim 1 wherein said image guiding stem is larger in diameter and longer in length than said light guiding stem.

6. The system according to claim 1 wherein said blade is permanently attached to said handle.

7. The system according to claim 6 further comprising a light guide and an image guide located within said blade.

8. The system according to claim 7 wherein said light guide and said image guide are permanently located within said blade.

9. A method for attaching a camera to a handle of a laryngoscope comprising the steps of:
providing a cavity within the handle;
providing a light guiding stem and an image guiding stem on the handle;
positioning a camera within the cavity, the camera having a light guiding receptacle and an image guiding receptacle complementary to and engagable with the light guiding stem and the image guiding stem;
engaging the light guiding stem and the image guiding stem with the light guiding receptacle and the image guiding receptacle respectively along a direction of movement.

10. A laryngoscope system comprising:
a handle having a first light guide and first image guide located therein;
a first light and image guiding connector associated with said handle;
a blade connected to said handle;
a second light guide and a second image guide located in said blade;
a second light and image guiding connector associated with said blade, said second light and image guiding connector being complementary to and engagable with said first light and image guiding connector along a direction of movement.

11. The laryngoscope system according to claim 10 further comprising a camera located within said handle, said camera generating an image signal.

12. The laryngoscope system according to claim 10 further comprising a video screen coupled to said camera for display of the image signal.

13. The laryngoscope system according to claim 10 wherein said blade is disposable.

14. The laryngoscope system according to claim 10 further comprising a power source located in said handle.

15. The laryngoscope system according to claim 10 further comprising an illuminating light source located in said handle.

16. The laryngoscope system according to claim 10 further comprising a locking device for securing said blade to said handle.

17. The laryngoscope system according to claim 10 wherein said second light guide and said second image guide are non-removably mounted within said blade.

18. The laryngoscope system according to claim 10 wherein said first light guide and said first image guide are in communication with said second light guide and said second image guide respectively, via the first and second light and image guiding connectors when in an engaged position.
